(19) United States
(12) Patent Application Publication
(10) Pub. No.: US 2022/0202986 A1
(43) Pub. Date: Jun. 30, 2022

(54) SANITARY ABSORBENT ARTICLE COMPRISING LACTOBACILLUS FORMULATION EFFECTIVE FOR DEODORIZATION

(71) Applicant: Hyuk Sun KWON, Seoul (KR)

(72) Inventor: Hyuk Sun KWON, Seoul (KR)

(21) Appl. No.: 17/610,019

(22) PCT Filed: Jul. 16, 2018
(86) PCT No.: PCT/KR2018/008013

§ 371 Date: Nov. 9, 2021

(30) Foreign Application Priority Data
Jul. 14, 2017 (KR) 10-2017-0089430

(51) Int. Cl. A61L 15/36; A61F 13/513; A61F 13/84; A61L 15/46; A61F 13/15

(52) U.S. Cl. CPC A61L 15/36; A61F 13/513; A61F 13/8405; A61F 2013/5109; A61F 13/15666; A61F 13/15804; A61L 15/46

(57) ABSTRACT

The present invention can provide a sanitary absorbent article which includes a Lactobacillus formulation inside an absorbent pad to absorb bodily secretions and effectively remove odor thereof without leakage of the Lactobacillus to the outside of the absorbent pad.

### TECHNICAL FIELD

The present invention relates to a sanitary absorbent article that can effectively remove the odor of bodily secretions by including a Lactobacillus formulation in an absorbent pad, and a method for manufacturing the same.

### BACKGROUND ART

Sanitary absorbent articles include sanitary pads for females, panty liners, tampons, incontinence articles, and baby diapers. These absorbent articles are commonly used to absorb and retain various secretions excreted from the human body. Since these secretions from the human body stink and cause discomfort, various methods have been developed to remove and reduce odor in absorbent articles.

As a known technique for removing odor from absorbent articles, a technique for applying a specific active material, which is activated when moisture comes in contact, to an absorbent article to remove odor has been disclosed (Korean Patent Laid-Open No. 10-1379049). However, there is a problem of reluctance to use chemical substances included in the absorbent article because the effect on the body cannot be considered to be good because these are located close to the body. Recently, a technique for removing odor from absorbent articles using a Lactobacillus formulation which is good for the body has been disclosed (Korean Patent Laid-Open No. 10-0581180). This technique uses wet spraying of a Lactobacillus formulation to place the Lactobacillus formulation inside an absorbent article and dry the same to a moisture content of less than 1% to produce the absorbent article, but in fact, it is not possible to confirm whether the Lactobacillus formulation can stably live: and be settled in the absorbent article during the drying process.

### RELATED ART DOCUMENTS

[Patent Document 1] Korean Patent Laid-Open No. 10-0581180
[Patent Document 2] Korean Patent Laid-Open No. 10-1379049

### DISCLOSURE

### Technical Problem

Accordingly, the present invention is directed to providing an absorbent article in which a Lactobacillus formulation is stably mounted on the absorbent article to effectively remove odor from bodily secretions without leaking from the absorbent article.

### Technical Solution

As such, the present invention is directed to providing an absorbent article in which a Lactobacillus formulation is stably mounted on an absorbent article to effectively remove odor from bodily secretions without leakage from the absorbent article.

In order to achieve the above object, the inventor of the present invention designed such that a Lactobacillus formulation is contained inside an absorbent pad that absorbs secretions, and in order to prevent the Lactobacillus formulation from leaking out of the absorbent article, the absorbent pad is entirely covered with a non-woven fabric such that the Lactobacillus formulation can be stably mounted inside the absorbent article. The absorbent article of the present invention has a structure in which the Lactobacillus formulation exists inside the absorbent pad to absorb secretions, remove odor from the secretions, and at the same time, prevent the Lactobacillus from escaping from the absorbent pad.

Specifically, an aspect of the present invention may provide a sanitary absorbent article for absorbing and retaining secretions discharged from the body through direct contact with the body, including (a) a liquid-permeable top sheet in direct contact with the skin, (b) an absorbent pad for absorbing and retaining secretions that have passed through the top sheet, (c) a liquid-impermeable cover sheet for preventing the secretions from leaking from the absorbent pad to the outside, and the absorbent pad includes a Lactobacillus formulation therein and a non-woven fabric for completely covering the absorbent pad. The absorbent article according to the present invention may provide an effect of removing or reducing the odor of secretions.

Herein, the absorbent pad is composed of three layers, the first and third layers are pulp layers composed of pulp, the second layer is an intermediate layer composed of a hydrogel, and in the second layer, the Lactobacillus formulation and a hydrogel are mixed.

In addition, another aspect of the present invention provides a method for manufacturing a sanitary absorbent article including a Lactobacillus formulation, and the method includes the following steps:

(i) mixing a Lactobacillus formulation and a hydrogel;

(ii) preparing an absorbent pad by placing pulp layers on the top and bottom with the mixture as a core layer;

(iii) compressing and drying the absorbent pad at a high temperature within about 1 second;

(iv) completely covering the absorbent pad including the Lactobacillus formulation with a non-woven fabric;

(v) disposing a liquid-permeable top sheet on the top of the absorbent pad covered with the non-woven fabric and disposing a liquid-impermeable back sheet on the bottom of the absorbent pad covered with the non-woven fabric; and

(vi) leaving a portion of the liquid-permeable top sheet in contact with the skin and covering the remaining three sides with a liquid-impermeable cover sheet to finish.

### Advantageous Effects

The absorbent article according to the present invention can effectively remove the odor of bodily fluids or secretions by stably containing a Lactobacillus formulation. In addition, since the absorbent article according to the present invention has a structure in which Lactobacillus does not leak to the outside, it does not affect the human body due to the leakage of Lactobacillus. In addition, the present invention has an advantage that sanitary absorbent articles including a

Lactobacillus formulation may be stored for a long period of time.

### DESCRIPTION OF DRAWINGS

FIG. 1 is a cross-sectional view of an absorbent article according to the present invention.

FIG. 2 is a cross-sectional view of an absorbent pad in the constitution of the absorbent article of the present invention.

FIG. 3 is a graph for quantifying the degree of odor in the absorbent article and the control group treated with a Lactobacillus formulation of Lactobacillus A or Lactobacillus B, respectively. Herein, Lactobacillus A is rhamnosus, and Lactobacillus B is acidophilus.

FIG. 4 is a graph for quantifying the level of H₂S in the absorbent article and the control group treated with a Lactobacillus formulation of Lactobacillus A or Lactobacillus B, respectively

FIG. 5 is a graph for quantifying the level of NH₃ in the absorbent article and the control group treated with a Lactobacillus formulation of Lactobacillus A or Lactobacillus B, respectively

FIG. 6 is an image for confirming whether Lactobacillus had leaked after leaving the absorbent article including a Lactobacillus formulation and the conventional absorbent article not including Lactobacillus for 4 days.

### MODES OF THE INVENTION

Hereinafter, terms used in the present specification will be described.

As used herein, the term “absorbent article” is used in a very broad sense to include any article capable of absorbing and retaining fluids or secretions, particularly, bodily secretions. An exemplary absorbent article related to the present invention is a disposable absorbent article. Absorbent articles suitable for use in the present invention include any type of structures ranging from a single absorbent pad structure to a complex multi-layer structure. Certain absorbent articles include a liquid-permeable top sheet, a back sheet which may be liquid-impermeable or permeable to water vapor or gas, and an absorbent pad which is also referred to as an absorbent core included therebetween.

As used herein, the term “disposable” is used in the present specification to mean an absorbent article that is not intended to be laundered or otherwise re-stored or reused as an absorbent article. Typical disposable absorbent articles according to the present invention include diapers, surgical and wound dressings and sweat pads, incontinence pads, absorbent articles for feminine hygiene such as sanitary pads, panty liners, tampons and the like.

As used herein, the term “secretion” includes sweat, urine, menstrual secretion, vaginal secretion and the like, but is not limited thereto, and it refers to any fluid or liquid produced and secreted by the body of animals, including humans.

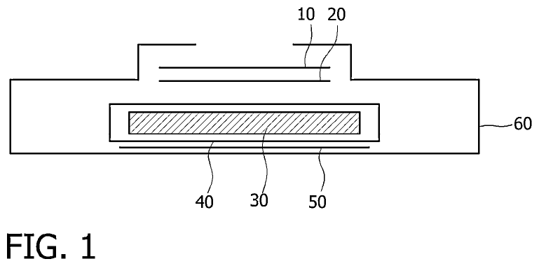

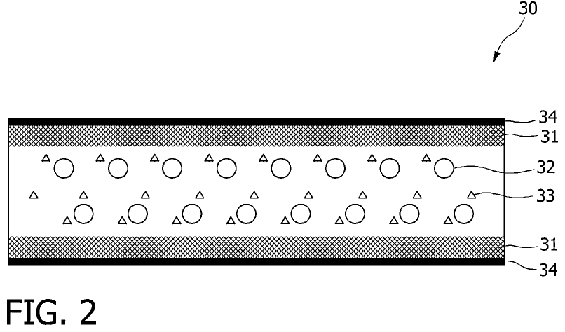

The configuration of the sanitary absorbent article according to the present invention may be described with reference to FIGS. 1 and 2.

The sanitary absorbent article according to the present invention is characterized in that while absorbing and retaining secretions separated from the human body, it includes a Lactobacillus formulation inside an absorbent pad to effectively remove the odor from the secretions. In particular, the present invention provides a structure in which the Lactobacillus formulation may be stably contained in an absorbent article.

Specifically, the present invention is a sanitary absorbent article for absorbing and retaining secretions discharged from the body through direct contact with the body, characterized with a structure including a liquid-permeable top sheet 10 in direct contact with the skin; an absorbent pad 30 for absorbing and retaining secretions that have passed through the top sheet; and a liquid-impermeable cover sheet 60 for preventing the secretions from leaking from the absorbent pad to the outside. Herein, the absorbent pad 30 includes a Lactobacillus formulation therein, and it is characterized in that a non-woven fabric having a plurality of micropores completely covers the absorbent pad 30 including the Lactobacillus formulation.

While the liquid-permeable top sheet 10 is a particular individual component of the absorbent article of the present invention including one or more layers, but in the absorbent article according to the present invention, the top sheet 10 refers to a layer in direct contact with the user's body during use.

The liquid-permeable top sheet 10 may be selected from woven and non-woven materials; polymeric materials such as thermoplastic films and plastic films; porous foam; reticular form; reticulated thermoplastic films; and thermoplastic scrims, but it may be manufactured from a wide range of materials which are not limited thereto.

The liquid-impermeable cover sheet 60 is impermeable to fluids or liquids, such as sweat, urine or menstrual secretions and vaginal secretions, and prevents such liquids or fluids from leaking out of the absorbent article. The liquid-impermeable cover sheet 60 may be made from a plastic film, for example, a microporous polyethylene film or a non-woven fabric. The cover sheet 60 is positioned adjacent to the outer facing surface of the absorbent pad 30. In addition, the cover sheet 60 serves to bond the absorbent article by any suitable attachment means known in the art. For example, the cover sheet 60 may fix the absorbent article by means of a uniform continuous layer of adhesive, a patterned layer of adhesive, or an array of discrete lines, spirals, or spots of adhesive.

One or several tissue layers 20 may be additionally positioned between the top sheet 10 and the absorbent pad 30. The tissue layer 20 is a liquid-permeable sheet. Accordingly, the bodily secretions may pass through the top sheet 10 and the tissue layer 20, and the absorbent pad 30 has a structure that may effectively absorb the secretions.

In addition, a back sheet 50 may be additionally positioned between the absorbent pad 30 and the cover sheet 60 thereunder. The back sheet 50 is a liquid-impermeable sheet.

Meanwhile, as shown in FIG. 1, the non-woven fabric 40 completely covers the absorbent pad 30. This structure may prevent the Lactobacillus formulation 33 contained in the absorbent pad 30 from leaking out of the absorbent article. The non-woven fabric 40 has micropores formed therein, and the size of the micropores may be about 0.1 μm to 1 μm.

FIG. 2 is a cross-sectional view of the absorbent pad 30 according to the present invention. Referring to FIG. 2, the absorbent pad 30 is composed of at least three layers, the first and third layers are pulp layers 31 composed of pulp, and the second layer located in the middle between the first layer and the third layer is a layer made of a hydrogel 32. The absorbent pad 30 has a structure in which a tissue layer 34 may be additionally positioned on the outer surfaces of the first layer and the third layer. The Lactobacillus formulation 33 is also included in the hydrogel layer inside the absorbent pad 30.

The Lactobacillus included in the absorbent pad 30 may have a concentration of 10⁶ to 10⁷ cfu. This is the amount of Lactobacillus included per one absorbent pad. The Lactobacillus formulation 33 is located inside the absorbent pad 30, that is, in the hydrogel 32 layer. The Lactobacillus formulation 33 is mixed with the hydrogel in the form of powder and is positioned inside the absorbent pad 30. The Lactobacillus formulation 33 is a mixture of Lactobacillus and auxiliary substances (e.g., starch powder and glutinous rice flour). The auxiliary substances serve as food for the Lactobacillus and help the Lactobacillus formulation 33 to be stably settled on the absorbent pad.

Lactobacillus which is the main ingredient of the Lactobacillus formulation (33) may be one or more selected from the Lactobacillus genus, the Lactococcus genus, the Enterococcus genus, the Streptococcus genus and the Bifidobacterium genus. The Lactobacillus genus may be one or more selected from the group consisting of L. acidophilus, L. fermentum, L. reuteri and L. rhamnosus. The Enterococcus genus may be E. faecium or E. faecalis. The Streptococcus genus may be S. thermophilus. The Bifidobacterium genus may be a species selected from the group consisting of B. bifidum. B. breve, B. longum, and B. animalis ssp. Lactis. Herein, the Lactobacillus genus is preferable as the Lactobacillus constituting the Lactobacillus formulation, and Lactobacillus acidophilus or rhamnosus species is more preferable. Herein, the absorbent article according to the present invention may be used by mixing one or more types of the Lactobacillus.

When the absorbent article of the present invention is a sanitary article for females such as a sanitary pad, a panty liner, or a mild incontinence article, this absorbent article is used as an attachment means and a means provided on the garment-facing surface of the cover sheet 60, and adhered to the crotch portion of the undergarment by a layer of pressure-sensitive adhesive, commonly referred to as an adhesive for fastening panties. Prior to use, the adhesive for fastening panties is protected by a release layer that releasably adheres to the adhesive, which is removed by the user when the absorbent article is to be attached to the undergarment to expose the adhesive. As known in the art, the release layer may be, for example, a sheet of silicone-treated paper, or a wrapper sheet typically made of a polymeric film, and the release layer may also provide be a releasable unitary wrapper for an absorbent article.

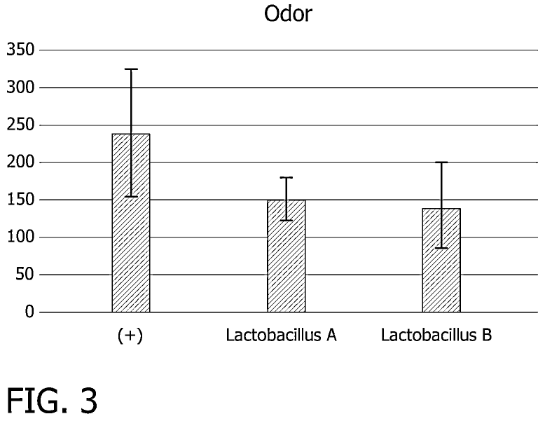

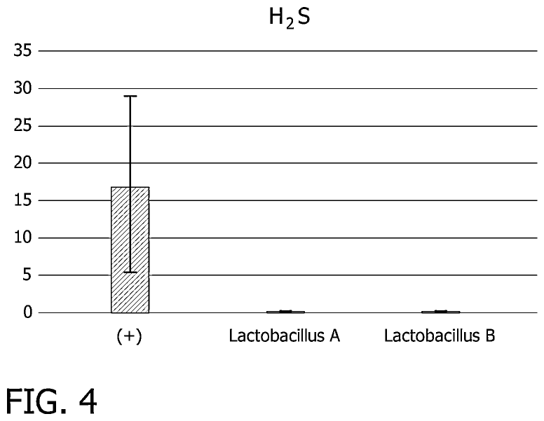

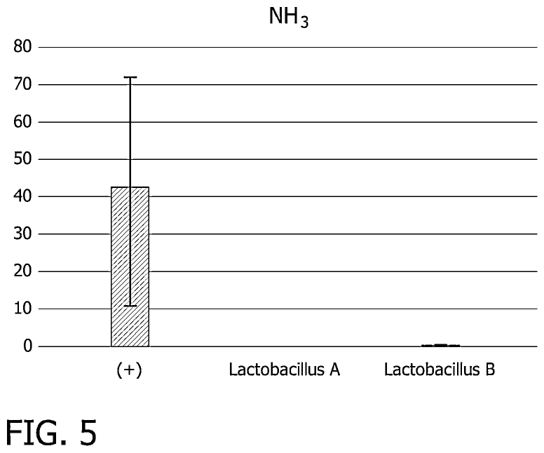

The sanitary absorbent article according to the present invention may remove the odor of secretions including blood such as menstrual blood, by including the Lactobacillus formulation inside the absorbent pad 30. In particular, it is very effective in removing gases such as odor, H₂S, and NH₃ from the decay of blood. The inventor of the present invention treated the Lactobacillus formulation inside the absorbent pad through an experiment, and in this case, the experiment was performed to measure the generation of odor, H₂S, and NH₃. It was confirmed that the generation of odor, H₂S, and NH₃ was effectively reduced in the absorbent article according to the present invention, compared to the control group (not including the Lactobacillus formulation) (refer to the graphs of FIGS. 3 to 5).

Further, in another aspect of the present invention, provided is a method for manufacturing an absorbent article including a Lactobacillus formulation, including the steps of:

(i) mixing a Lactobacillus formulation and a hydrogel;

(ii) preparing an absorbent pad by placing pulp layers on the top and bottom with the mixture as a core;

(iii) compressing and drying the absorbent pad at a high temperature within about 1 second;

(iv) completely covering the absorbent pad including the Lactobacillus formulation with a non-woven fabric;

(v) disposing a liquid-permeable top sheet on the top of the absorbent pad covered with the non-woven fabric and disposing a liquid-impermeable back sheet on the bottom of the absorbent pad covered with the non-woven fabric; and

(vi) leaving a portion of the liquid-permeable top sheet in contact with the skin and covering the remaining three sides with a liquid-impermeable cover sheet to finish.

Herein, in step (iii), the absorbent pad may be compressed by applying heat at 135° C.

As described above, the manufacturing method according to the present invention provides a structure in which the Lactobacillus formulation may be stably settled in the absorbent article. In particular, since the non-woven fabric 40 has a structure that completely covers the outside of the absorbent pad 30 and the non-woven fabric has fine pores, it is possible to prevent the Lactobacillus formulation 33 from leaking from the absorbent article.

### EXAMPLE 1

### Manufacture of Absorbent Articles Including Lactobacillus Formulation

(1) Preparation of Lactobacillus formulation: Mix Lactobacillus and starch powder at a ratio of 1:1 to include 10⁶ to 10⁷ cfu of Lactobacillus per one absorbent pad.

(2) Mixing of Lactobacillus formulation and a hydrogel: Mix the Lactobacillus formulation and a hydrogel to prepare as a core material of the absorbent pad.

(3) Preparation of absorbent pad: The absorbent pad was prepared in a sandwich structure with pulp layers on the outside and a hydrogel mixture as a core in the middle. The absorbent pad structure was heat treated at 135° C. for about 1 second, and the absorbent pad was compressed and dried.

(4) The prepared absorbent pad was completely covered with a non-woven fabric and then compressed at room temperature to adhere the non-woven fabric to the absorbent pad. The Lactobacillus formulation was prepared with Lactobacillus A and Lactobacillus B, respectively.

Herein, Lactobacillus A is rhamnosus, and Lactobacillus B is acidophilus. Afterwards, it was prepared according to a conventional method for manufacturing an absorbent article.

### EXAMPLE 2

### Comparison Experiment of Odor Removal Performance

After preparing bovine glands, these were mixed with a mixer, and 10 mL of the mixed glands (Lactobacillus formulation treatment group 100 mg Lactobacillus formulation/10 mL) was loaded per one absorbent article. This loading amount corresponds to the average volume of menstrual blood. The absorbent article loaded with the glands was placed in a plastic bag and sealed to prevent gas and odor from escaping during the reaction process. The sealed absorbent article was placed in a 37° C. incubator for 24 hours. The sensor of a measuring instrument was plugged into the inside of the plastic bag and the odor, NH₃, and H₂S were measured. Calibration was always performed before measurement. When measuring odor, NH₃, and H₂S, in order to prevent errors due to air dilution during measurement, the sealing vinyl was drilled only to the extent that it was possible for the equipment to enter. The comparison experiment was repeated 5 times for one item and summarized in Table 1 below, and by showing the average values of the control group and Lactobacillus A and B for odor, NH₃, and H₂S, respectively, the graphs of FIGS. 3, 4 and 5 numerically show the degree of odor removal with and without the Lactobacillus formulation.

TABLE 1

| Classifi- | Odor | | | | | | H₂S | | | | | | NH₃ | | | | | |
|---|---|---|---|---|---|---|---|---|---|---|---|---|---|---|---|---|---|---|
| cation | S1 | S2 | S3 | S4 | S5 | AVR | S1 | S2 | S3 | S4 | S5 | AVR | S1 | S2 | S3 | S4 | S5 | AVR |
| (+) | 171 | 215 | 329 | — | — | 238.33333 | 8.38 | 30.27 | 12.08 | — | — | 16.91 | 18.86 | 75.35 | 32.75 | — | — | 42.32 |
| Lactoba- | 148 | 117 | 171 | 190 | 137 | 152.6 | 0.48 | 0.22 | 3.7 | 0.03 | 0.18 | 0.256 | 0 | 0 | 0 | 0 | 0 | 0 |
| cillus A | | | | | | | | | | | | | | | | | | |
| Lactoba- | 223 | 75 | 105 | 150 | 162 | 143 | 0.36 | 0.09 | 0.29 | 0.42 | 0.48 | 0.324 | 0.29 | 0.18 | 0.12 | 0.37 | 0.46 | 0.284 |
| cillus B | | | | | | | | | | | | | | | | | | |

### EXAMPLE 3

### Confirmation of External Leakage of Lactobacillus

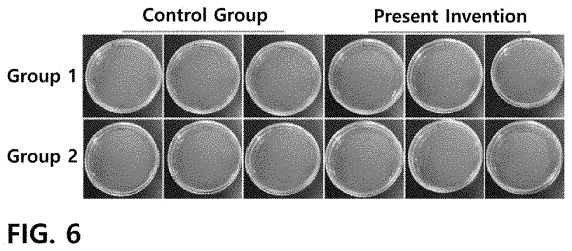

A total of seven absorbent articles according to the present invention were prepared. These absorbent articles were products manufactured according to Example 1 and included the Lactobacillus formulation. Accordingly, a total of 7 general absorbent articles (control group) that did not include Lactobacillus were prepared. Three absorbent articles according to the present invention containing Lactobacillus and three control groups were left at room temperature (Group 1). Three absorbent articles containing Lactobacillus according to the present invention and three control groups were left at 37° C., which is similar to body temperature, for 4 hours (Group 2). Boiled and cooled tertiary distilled water was prepared 60 times the weight of the absorbent article. The absorbent articles of Groups 1 and 2 were reacted with the prepared tertiary distilled water for 5 minutes. 10 mL of the reacted solution was transferred to a 15 mL test tube. The solution reacted with each sample was buried in a solid medium dedicated to culturing Lactobacillus and smeared. The Lactobacillus-plated solid medium plate was incubated at 37° C. for 48 hours. The growth of bacteria was confirmed. As shown in FIG. 6, no Lactobacillus colony was identified.

### INDUSTRIAL APPLICABILITY

The absorbent article according to the present invention includes a Lactobacillus formulation, but Lactobacillus does not leak to the outside of the absorbent article such that only odor can be effectively reduced without affecting the human body.

1. A sanitary absorbent article for absorbing and retaining secretions discharged from the body through direct contact with the body, comprising (a) a liquid-permeable top sheet in direct contact with the skin, (b) an absorbent pad for absorbing and retaining secretions that have passed through the top sheet, (c) a liquid-impermeable cover sheet for preventing the secretions from leaking from the absorbent pad to the outside, and a non-woven fabric for completely covering the absorbent pad,
wherein the absorbent pad comprises a Lactobacillus formulation therein.

2. The sanitary absorbent article of claim 1, wherein the absorbent pad is composed of three layers, the first and third layers are pulp layers composed of pulp, the second layer is an intermediate layer composed of a hydrogel, and in the second layer, the Lactobacillus formulation and a hydrogel are mixed.

3. The sanitary absorbent article of claim 1, wherein Lactobacillus which is the main ingredient of the Lactobacillus formulation is one or more selected from the Lactobacillus genus, the Lactococcus genus, the Enterococcus genus, the Streptococcus genus and the Bifidobacterium genus.

4. The sanitary absorbent article of claim 3, wherein the Lactobacillus genus is selected from the group consisting of L. acidophilus, L. fermentum, L. reuteri and L. rhamnosus.

5. The sanitary absorbent article of claim 1, wherein the Lactobacillus formulation comprises Lactobacillus acidophilus or Lactobacillus rhamnosus.

6. The sanitary absorbent article of claim 1, wherein the amount of the Lactobacillus is 10⁶ to 10⁷ cfu.

7. A method for manufacturing a sanitary absorbent article comprising Lactobacillus, the method comprising:
(i) mixing a Lactobacillus formulation and a hydrogel;
(ii) preparing an absorbent pad by placing pulp layers on the top and bottom with the mixture as a core;
(iii) compressing and drying the absorbent pad at a high temperature within about 1 second;
(iv) completely covering the absorbent pad with a non-woven fabric;
(v) disposing a liquid-permeable top sheet on the top of the absorbent pad covered with the non-woven fabric and disposing a liquid-impermeable back sheet on the bottom of the absorbent pad covered with the non-woven fabric; and
(vi) leaving a portion of the liquid-permeable top sheet in contact with the skin and covering the remaining three sides with a liquid-impermeable cover sheet to finish.

8. The method of claim 7, wherein the absorbent pad is compressed and dried by applying heat at 135° C. in step (iii) above.

9. The method of claim 7, wherein Lactobacillus which is the main ingredient of the Lactobacillus formulation is one or more selected from the Lactobacillus genus, the Lactococcus genus, the Enterococcus genus, the Streptococcus genus and the Bifidobacterium genus.

10. The method of claim 7, wherein the amount of the Lactobacillus is 10⁶ to 10⁷ cfu.
